(19) United States
(12) Patent Application Publication
(10) Pub. No.: US 2026/0125492 A1
(43) Pub. Date: May 7, 2026

(54) ENZYMATIC GRAPHENE-PEPTIDE DISPERSION

(71) Applicant: Research Foundation of the City University of New York, New York, NY (US)

(72) Inventors: Kenny Barriales, New York, NY (US); Shadman Kandaker, New York, NY (US); Ankit Jain, New York, NY (US); Rein Ulijn, New York, NY (US)

(21) Appl. No.: 18/927,559

(22) Filed: Oct. 25, 2024

Related U.S. Application Data
(63) Continuation-in-part of application No. 18/805,215, filed on Aug. 14, 2024, now abandoned.
(60) Provisional application No. 63/519,402, filed on Aug. 14, 2023.

(51) Int. Cl. C07K 17/14; C12N 9/02; C12P 3/00; C12P 21/02

(52) U.S. Cl. CPC C07K 17/14; C12N 9/0071; C12P 3/00; C12P 21/02; C12Y 114/18001

(57) ABSTRACT

A method for forming an aqueous graphene-peptide dispersion. Graphene, a peptide, water and a tyrosinase enzyme are mixed. The resulting suspension is sonicated or vortexed until an aqueous graphene-peptide dispersion forms.

### CROSS-REFERENCE TO RELATED APPLICATIONS

This application claims priority to, and is a continuation-in-part of, U.S. patent application Ser. No. 18/805,215 (filed Aug. 14, 2024) which is a non-provisional of, U.S. Patent Application 63/519,402 (filed Aug. 14, 2023), the entirety of which are incorporated herein by reference.

### STATEMENT OF FEDERALLY SPONSORED RESEARCH OR DEVELOPMENT

This invention was made with government support under grant number FA9550-21-1-0091 awarded by the Air Force Office of Scientific Research. The government has certain rights in the invention.

### BACKGROUND OF THE INVENTION

Graphene, a two-dimensional carbon material, possesses extraordinary mechanical, electrical, and thermal properties, making it highly attractive for various biological applications such as biosensing, biotherapeutics and tissue engineering. However, the tendency of graphene sheets to aggregate and restack hinders its dispersion in water, limiting its potential applications. An improved method for dispersing graphene in water is therefore desired.

The discussion above is merely provided for general background information and is not intended to be used as an aid in determining the scope of the claimed subject matter.

### SUMMARY

This disclosure provides a method for forming an aqueous graphene-peptide dispersion.

In a first embodiment, a method for forming an aqueous graphene-peptide dispersion is provided. The method comprising: mixing graphene, a peptide having from two to four residues, water and an oxidant, the peptide having a first residue that is Y, a second residue that is H, Y, W, F, A, V, L, I, P or M, optionally a third residue that is K, H, R, D or E and optionally a fourth residue that is an amino acid, the peptide having an N-terminus that is optionally acetylated and a C-terminus that is optionally amidated, thereby forming a suspension; and sonicating or vortexing the suspension until an aqueous graphene-peptide dispersion forms.

In a second embodiment, a method for forming an aqueous graphene-peptide dispersion is provided. The method comprising: mixing graphene, a tripeptide, water and an oxidizing enzyme, the tripeptide having a first residue that is Y, a second residue that is H, Y, W, F, A, V, L, I, P or M, and a third residue that is K, H, R, D or E, the tripeptide having an N-terminus that is optionally acetylated and a C-terminus that is optionally amidated, thereby forming a suspension; and sonicating or vortexing the suspension until an aqueous graphene-peptide dispersion forms.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1A is a schematic representation of KYF dispersing graphene sheets and further dispersion through enzymatic oxidation.

FIG. 1B illustrates a molecular structure of KYF and radical formation structure after tyrosinase is introduced.

FIG. 1C shows illustrations of the peptide structure covalently bound onto the graphene surface with corresponding covalent and non-covalent interactions (top) top-down view to emphasize x-stacking (bottom). Hydrogens were removed for clarification.

FIG. 1D depicts macroscopic and Cryo-TEM images of 1G, 2G and 3G respectively. Scale bar=500 nm. Inset Scale bar=200 nm

FIG. 2A show optical Images of 1G, 2G and 3G. Scale bar=200 μm.

FIG. 2B depicts a zeta potential analysis of 1G, 2G and 3G (n=3).

FIG. 2C shows a UV-V absorption spectra in the broad absorption range (250-800 nm).

FIG. 2D shows SEM micrographs of 1G, 2G and 3G. Scale bar=50 μm.

FIG. 3A is a Raman spectroscopy of 1G, 2G and 3G with corresponding D, G and 2D band values.

FIG. 3B is a bar graph showing the I_{D}/I_{G} ratio of each sample; 0.47, 0.34 and 0.76 for 1G, 2G and 3G, respectively.

FIG. 3C is a bar graph showing the I_{2D}/I_{G} ratio of each sample; 0.42, 0.74 and 0.75 for 1G, 2G and 3G, respectively.

FIG. 3D depicts XPS C 1s, N 1s and O 1s spectra. Composite spectra are shown for C 1s and O 1s to eliminate noise for clarification, raw spectra are shown for N 1s.

FIG. 4A shows a step-by-step representation of the purification process to remove enzymes, unreacted peptides, salts, and other impurities of 3G.

FIG. 4B shows an optical image of 3G after redispersion, Scale bar=200 μm.

FIG. 4C illustrates macroscopic images of the final product redispersed in water over the time course of 15 s.

FIG. 5 shows the results of liquid chromatography-mass spectrometry of KYF tripeptide.

FIG. 6 is a UV-visible absorbance spectra for 3G showing change in absorbance 1 hour after sample was collected and 5 weeks after sample was collected.

FIG. 7 shows an electron paramagnetic resonance (EPR) spectra of KYF and KYF_ox.

FIG. 8 depicts a zeta Potential values of all mentioned control experiments. All samples contain nano graphene with the exception of p-G (KYF ox) as this contains pristine graphene. 1G, 2G and 3G were provided here for easy comparison. (Ac=Acetylated, ox=Oxidized).

FIG. 9 is a UV-visible absorbance spectra for inactivated enzyme controls done after raising pH to 12 (brown line) and heat to 120° C. (orange line).

FIG. 10 is a UV-visible absorbance spectra result for peptide-graphene stoichiometry study at 0.5 mM, 2 mM, and 5 mM peptide concentrations.

FIG. 11 shows TGA data showing the weight loss over a function of temperature of the peptide KYF and 3G after subjected to an ethanol wash procedure.

FIG. 12 illustrates a comparison control experiment showing surfactants best known to dispersed graphene sodium dodecyl sulfate (SDS), sodium dedecylbenzenesulfonate (SDBS), and cetrimonium bromide (CTAB). Macroscopic images after 1 and 72 hours of settlement in room temperature (20-30° C.). Bottom row shows 3G after one month of settlement in room temperature (20-30° C.).

FIG. 13 is a schematic of the ethanol wash procedure for 3G

FIG. 14 illustrates XPS C 1s, N 1s, O 1s and Si 2p spectra shown for 1G, 2G and 3G. Measured spectra are presented with bold lines, while their corresponding fitted envelopes are shown for each sample. Raw Spectra is shown for nitrogen. Deconvolution is shown for the C 1s spectra in gray.

FIG. 15 shows a copper assay showing the complete removal of the metallo-enzyme after proceeding the ethanol washing step.

FIG. 16 depicts eta Potential Measurements of all dipeptides mentioned.

### DETAILED DESCRIPTION OF THE INVENTION

This disclosure provides a method for dispersing graphene in water and a composition resulting from the method. Graphene is covalently functionalized with an amphiphilic dipeptide, tripeptide or tetrapeptide through the tyrosine sidechain via oxidation. In one embodiment the tripeptide consists of a first residue that is Y, a second residue that is H, Y, W, F, A, V, L, I, P or M and a third residue that is K, H, R, D or E. In one embodiment, the second residue is selected from H, Y, W or F. In another embodiment, the second residue is selected from A, V, L, I, P or M. The reference to the first residue, the second residue and third third residue is merely to distinguish each residue and does not indicate the relative positioning of the residues. For example, the first residue (Y) may appear in the first position, the second position or the third position in the tripeptide. Examples demonstrated include KYF, HYF, KYY, HYY, DYF, EYF and Ac-KYF, Ac-KYF-NH₂, KYF-NH₂ derivatives. In another embodiment, the tetrapeptide consists of four amino acids including a first residue that is tyrosine (Y), a second residue selected from H, Y, W, F, A, V, L, I, P or M, a third residue selected from K, H, R D or E and a fourth residue that may be any amino acid. The dipeptide consists of two amino acids including a first residue that is tyrosine (Y) and a second residue that is H, Y, W, F, A, V, L, I, P or M. In the aforementioned embodiments the N-terminus may optionally undergo acetylation and/or the C-terminus may optionally undergo amidation. Examples of suitable oxidants include tyrosinase enzymes, photooxidation with ultraviolet light, reactive oxygen species (e.g. peroxides including hydrogen peroxide) with or without metal catalysts (e.g. copper) and electrochemical oxidation.

This disclosure provides a biocompatible method of dispersing, exfoliating and functionalizing graphene in aqueous media that is suitable for biomedical materials and sensing applications. The functionalization method allows graphene to be readily dispersed in aqueous media and the material can be lyophilized, and readily re-dispersed.

The disclosure provides a biocompatible material that reduces or eliminates the restacking and aggregation tendency that graphene has in water. Through this biofunctionalization method, an array of possibilities emerge, where different peptides sequences containing tyrosine can finely tune and modify the functionality of graphene, enabling the integration into a toxic free and biocompatible range of applications. The reproducibility and simplicity of this process allows one to produce pre-functionalized graphene products that are dispersible in water, competing with commonly utilized graphene analogues such as graphene oxide (GO) or reduced graphene oxide r-GO. In theory, given the diversity of function that arise from different peptide sequences, covalent modification using this methodology allows for one to adjust the residues used based on application needs.

This disclosure provides a method for covalently functionalizing graphene with an amphiphilic tripeptide (KYF), facilitated by the tyrosine phenol side chain, through an enzymatic oxidation process. The presence of phenylalanine (F) enhances this interaction through non-covalent support via π-π stacking with the graphene surface. Lysine (K) involvement enables effective interaction with water molecules, resulting in the dispersion of the newly functionalized graphene in aqueous solutions.

In the following examples, an amphiphilic tripeptide, KYF (lysine-tyrosine-phenylalanine), and several related cationic and anionic tripeptides are used, which combine aromatic residues that stack with the graphene lattice, and a cationic residue to facilitate aqueous dispersion. The use of an oxidizing enzyme plays a catalytic role in the functionalization process, as it generates free radicals on the hydroxy group of the tyrosine and interacts directly onto the sp² lattice. In another embodiment, the oxidation occurs photochemically through the application of ultraviolet (UV) light. Subsequently, under prolonged (e.g. 24-hour) sonication, the peptide molecules undergo covalent bonding with graphene through the tyrosine sidechain. Sonication generally continues for at least one hour. In one embodiment, sonication continues for at least six hours. In another embodiment, sonication continues for at least twelve hours. In one embodiment, sonication is replaced with vortexing. This reaction is aided by a hydrophobic amino acid sidechain group, which provides non-covalent support to the graphene surface via π-π stacking.

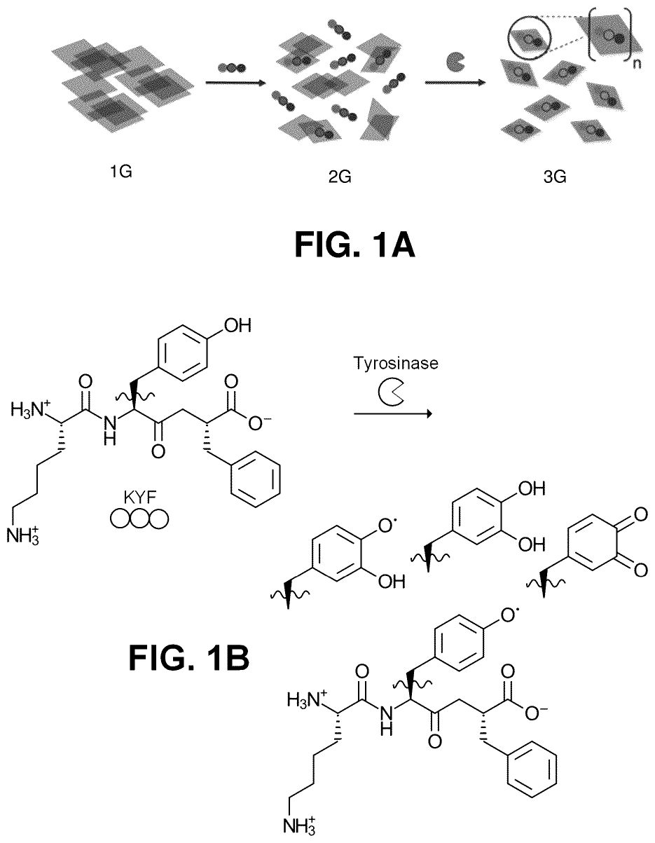

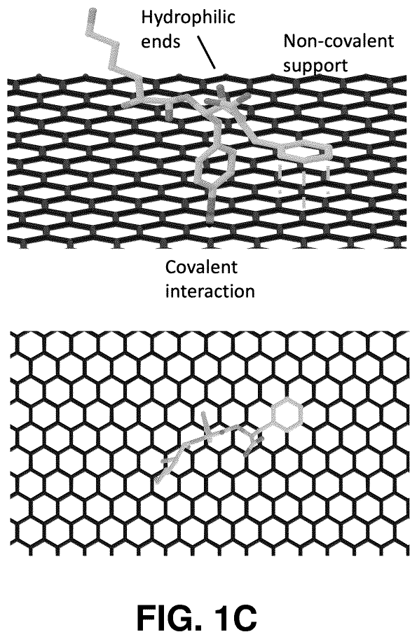

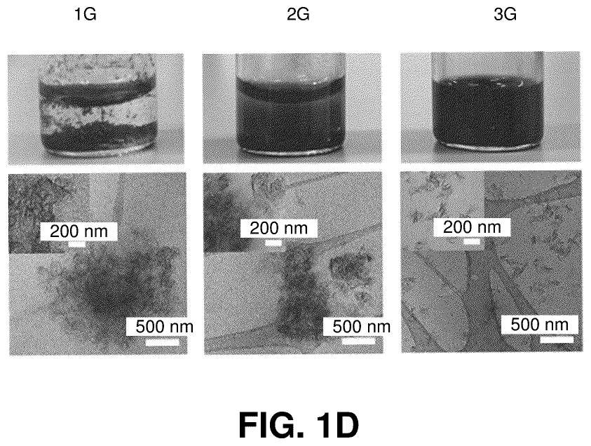

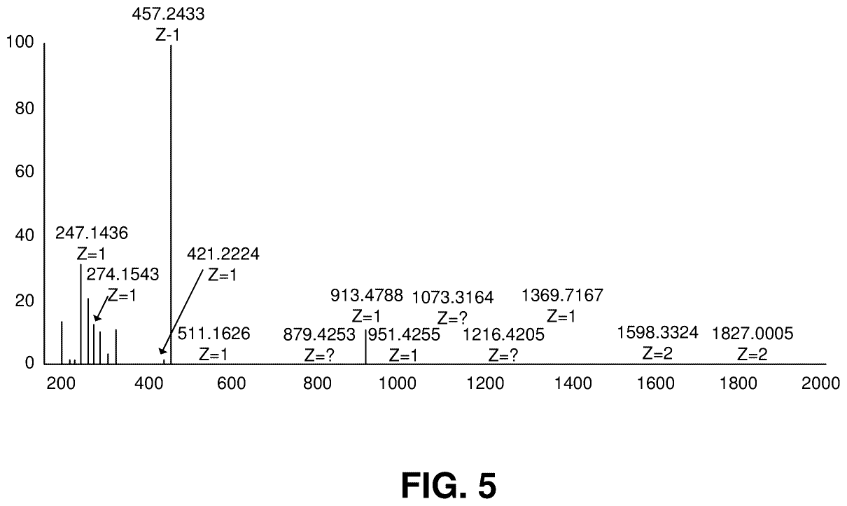

Graphene (G) nanosheets immediately aggregate in water and cannot be re-dispersed even after 24 hours of rigorous bath sonication (1G, FIG. 1A and FIG. 1D). Similarly, the addition of amphiphilic, aromatic KYF peptide to the graphene (LCMS shown, FIG. 5) at 2 mM prior to sonication results only in a low level of dispersion as consequence of the intermolecular interactions of two aromatic amino acids n-stacking with the surface of graphene (2G, FIG. 1A and FIG. 1D). Introduction of KYF and 0.2 mg mL⁻¹ mushroom tyrosinase, followed by 24 hours of bath sonication, resulted in a dispersed solution that is macroscopically detectable and exhibits remarkable stability (3G, FIG. 1A and FIG. 1D).

TABLE 1

| Graphene | |
|---|---|
| Sample | Treatment |
| 1G | Graphene after 24 hours of rigorous bath sonication |
| 2G | Graphene after 24 hours of rigorous bath sonication |
| | with 2 mM KYF peptide |
| 3G | Graphene after 24 hours of rigorous bath sonication |
| | with 2 mM KYF peptide and 0.2 mg mL⁻¹ tyrosinase |

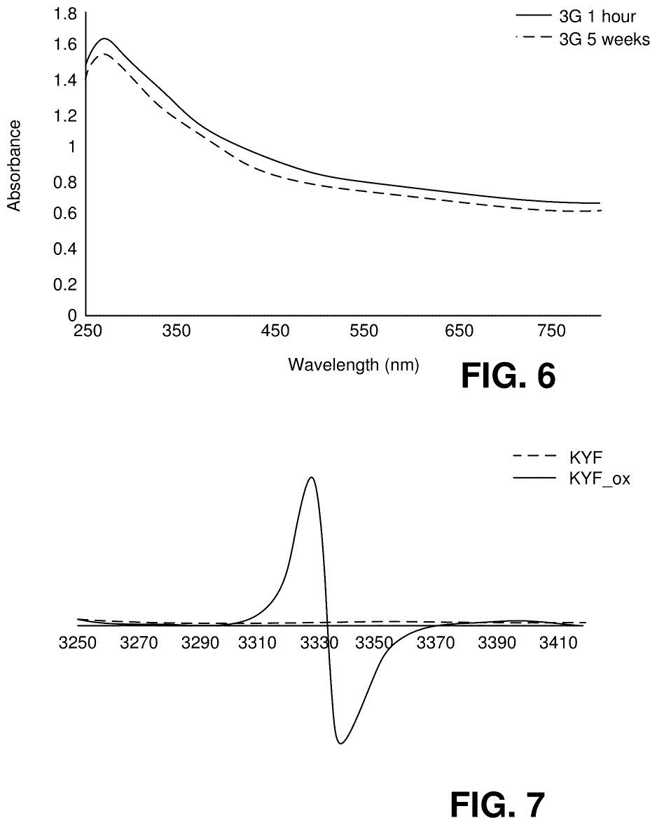

Other concentrations of peptide and enzyme may be utilized to facilitate dispersion. For example, the peptide may be present in a concentration of between 0.1 mM and 100 mM. In another embodiment, the peptide is present in a concentration of between 0.5 mM and 10 mM. In yet another embodiment, the peptide is present in a concentration between 0.5 and 5 mM. Similarly, the enzyme may be present in a concentration between 0.01 mg per mL and 1 mg per mL. In another embodiment, the enzyme is present in a concentration between 0.1 mg per mL and 1 mg per mL. Other temperatures may be utilized provided the tyrosinase enzyme is active. In some embodiments, a pH other than 7.4 is used provided the pH is between 5-9. This dispersion is shown to be stable for many weeks with little fluctuations of the scattered absorbance measured by UV-vis spectroscopy (FIG. 6). The catalytic role of the enzyme becomes evident in the functionalization process as the enzyme effectively facilitates the oxidation of KYF, resulting in the generation of free radicals on the hydroxy group of the tyrosine (FIG. 1B), leading to approximately 90% of the graphene being functionalized. Besides the radical phenol, it is likely that other enzymatic reaction products, like the 3,4-dihydroxyphenylalanine (L-DOPA) and its catechol and corresponding radical oxidation products contribute (FIG. 1B). Evidence of this free radical production after oxidation is supported by Electron Paramagnetic Resonance (EPR) to detect the presence of radical formation (FIG. 7). These radicals directly interact with the sp² lattice of graphene giving rise to a covalent bonding interaction aided by the non-covalent support of the phenylalanine (F) while the hydrophilic amine group on the lysine (K) and terminal groups interact with water molecules for a stable dispersion (FIG. 1C). A top-down view of the peptide-graphene interaction is shown to emphasize the π-stacking of the phenylalanine (F) (FIG. 1C). To provide a comprehensive qualitative assessment of the system, the macroscopic and cryo-transmission electron microscopy (cryo-TEM) techniques were employed (FIG. 1D). In the case of 1G, notable clumping and aggregation was observed, indicating a lack of dispersion. For 2G, a partial dispersion of the graphene clumps was evident; however, cryo-TEM images still exhibited some residual aggregation. Conversely, the images of 3G revealed a visually uniform, complete black solution, indicative of a high degree of dispersion. Notably, cryo-TEM displayed distinct separation and dispersion between graphene flakes in 3G, emphasizing the effectiveness of the dispersal process.

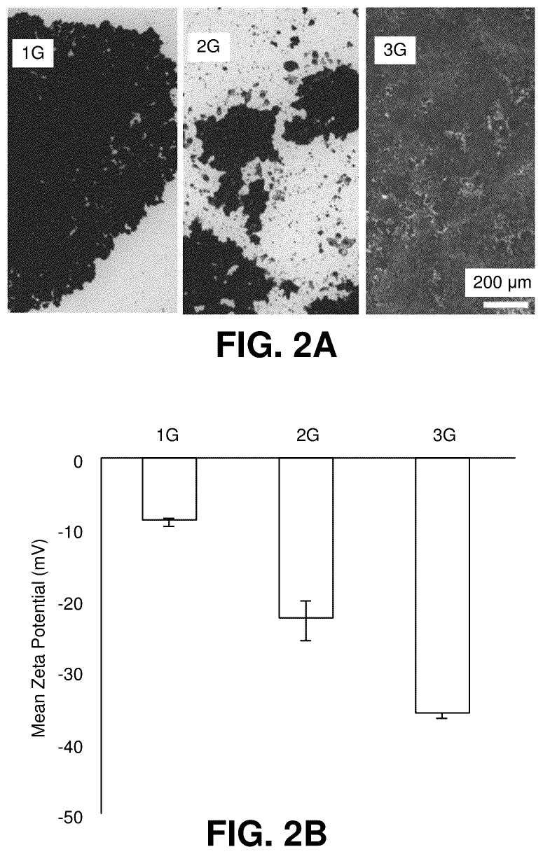

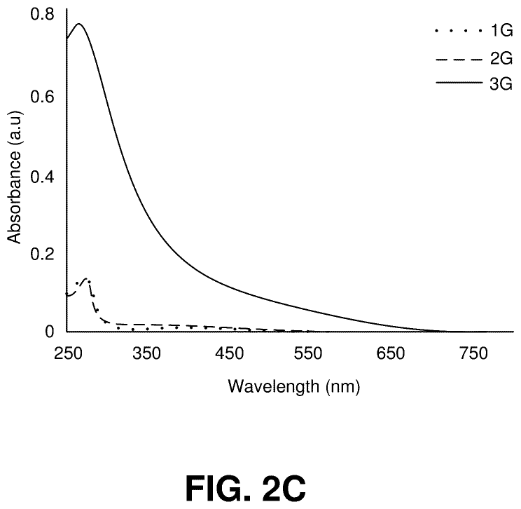

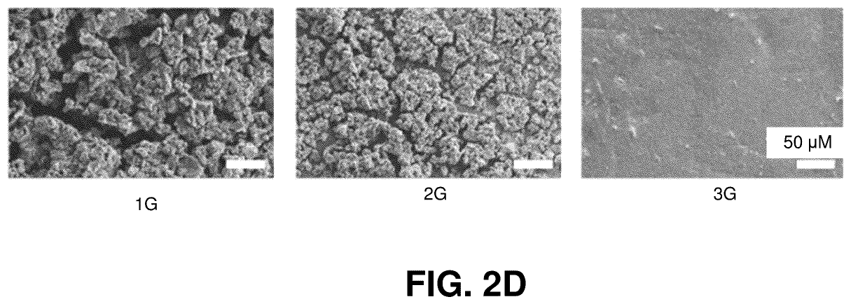

To validate dispersion and stability of the system as shown through optical microscopy (FIG. 2A) the role of electrostatic stabilization was measured via zeta potential analysis (FIG. 2B). Zeta potential analysis serves to assess repulsive and attractive forces present among graphene sheets suspended within the liquid medium. In the context of dispersion, nanoparticles are considered stable when the absolute values of the zeta potential exceed the threshold of ±30 mV. A value of −35.9 mV was obtained for 3G showing that 3G is a stable graphene dispersion. In contrast, 1G recorded a value of −8.7 mV and 2G recorded a value of −22.5 mV, showing that 1G and 2G are unstable graphene dispersions (FIG. 2B).

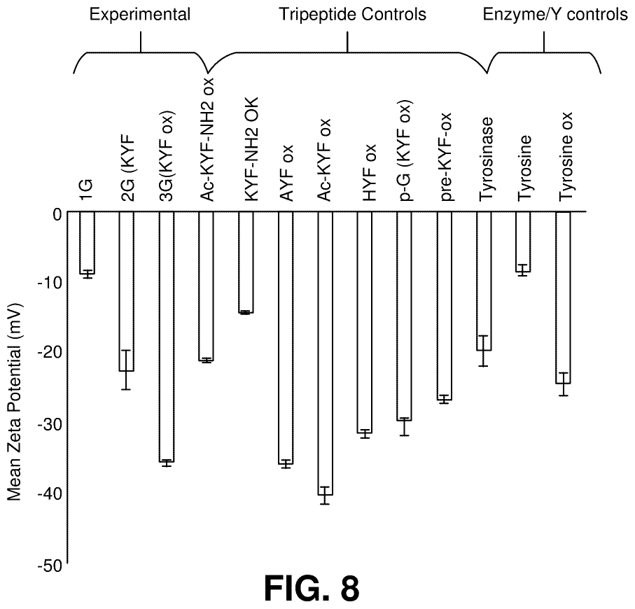

Additional control experiments were conducted to elucidate and verify the structural conformation of the peptide. The peptide derivatives N-Ac-KYF (−40.6 mV) and KYF-NH₂ (−14.2 mV) were used to test the effect of the N- and C-terminus, as well as Ac-KYF-NH₂ (−21.2 mV), while AYF (−35.9 mV) served to study the removal of the lysine side chain (FIG. 8). These control experiments highlighted the significant role of the ionizable groups that are responsible for aqueous dispersion. Without wishing to be bound to any particular theory, the main dispersing group is believed to be the terminal carboxylate, and the cationic groups may participate in both dispersion interactions and that of the graphene sp² lattice structure.

Experimental controls involving the single amino acid tyrosine (Y) with graphene in presence of tyrosinase were performed to validate the use of a second aromatic group and additional ionizable group for the peptide to adopt a stable conformation on the graphene surface. The zeta potential measurements for both did not exceed −30 mV (FIG. 8). in order to test if the enzyme itself plays a role in dispersion, tyrosinase (Y) alone with graphene was recorded with a value below −20 mV, providing confirmation that no undesirable interactions with the enzyme and the carbon material occurred during functionalization (FIG. 8). The combination of pre-oxidized KYF with graphene did not yield a stable dispersion due to the polymerization of the oxidized peptide, leading to the formation of oligomers as previously reported, thereby hindering active radicals from effectively attacking the sp² carbons on the graphene surface (FIG. 8).

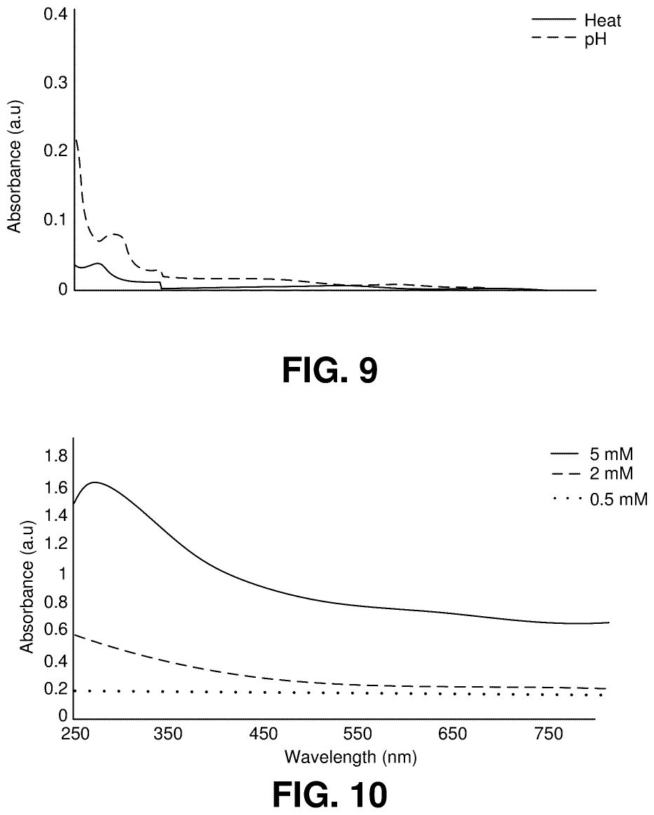

In order to further verify the occurrence of enzymatic peptide oxidation and consequent dispersion of the graphene sheets, UV-visible spectroscopy was utilized. 1G showed no observable peaks while 2G showed presence of a tyrosine peak at 270 nm. 3G showed a broad absorption due to scattering from the graphene sheets, confirming their uniform dispersion. Some characteristic oxidation signatures of tyrosine in solution can be observed through absorption throughout the visible region (420 to 650 nm) (FIG. 2C). Two control experiments, where (1) the enzyme was inactivated by heating at 120° C. for 30 min and (2) wherein the enzyme was inactivated by changing the pH to 12 were conducted to assess the enzymatic activity in initiating the oxidation reaction for effective dispersion (FIG. 9). These control samples exhibited no discernible UV-absorption signals and failed to attain stable zeta potential values, underscoring the importance of an oxidant in facilitating graphene dispersion. To test for optimal peptide-graphene stoichiometry UV-vis was used to grasp the maximum absorbance of scattered graphene under three different peptide concentrations 0.5 mM, 2 mM and 5 mM (FIG. 10). Without wishing to be bound to any particular theory, at high concentrations of peptide, the molecules are believed to interact with each other more in favor of creating oligomers, while at low concentrations there is insufficient peptide material to coat the graphene surface to produce stable dispersions.

Scanning Electron Microscopy (SEM) was employed to examine the morphology of functionalized graphene, after drying at room temperature (25° C.) overnight. Consistent with the cryo-TEM observations, SEM images revealed distinct characteristics of the three samples. Specifically, while 1G and 2G exhibited evident clumping as a result of graphene aggregation, in contrast, sample 3G shows a uniform surface, thus confirming the successful dispersion and negating the tendency to restack via VdW forces (FIG. 2D).

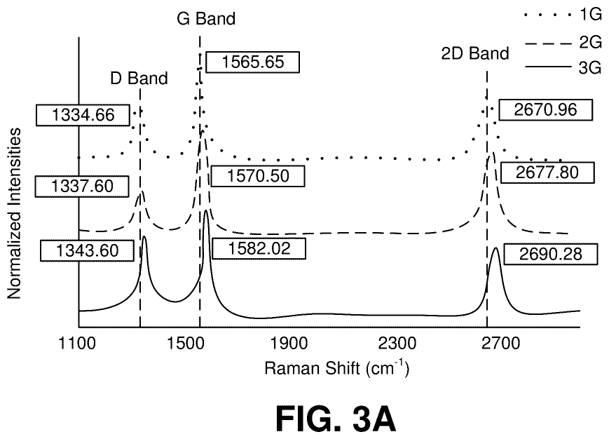

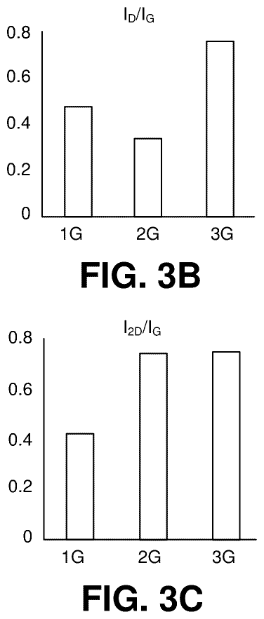

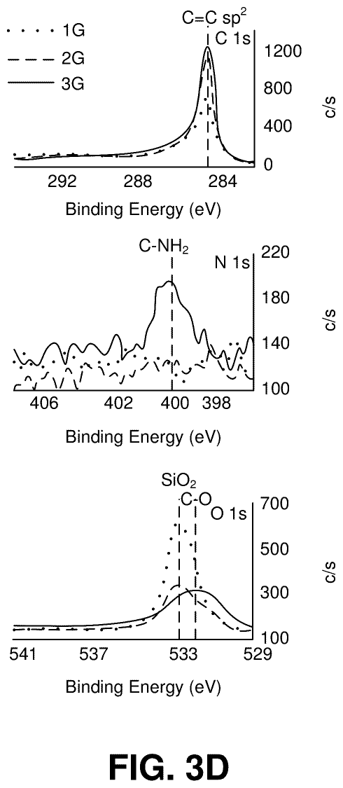

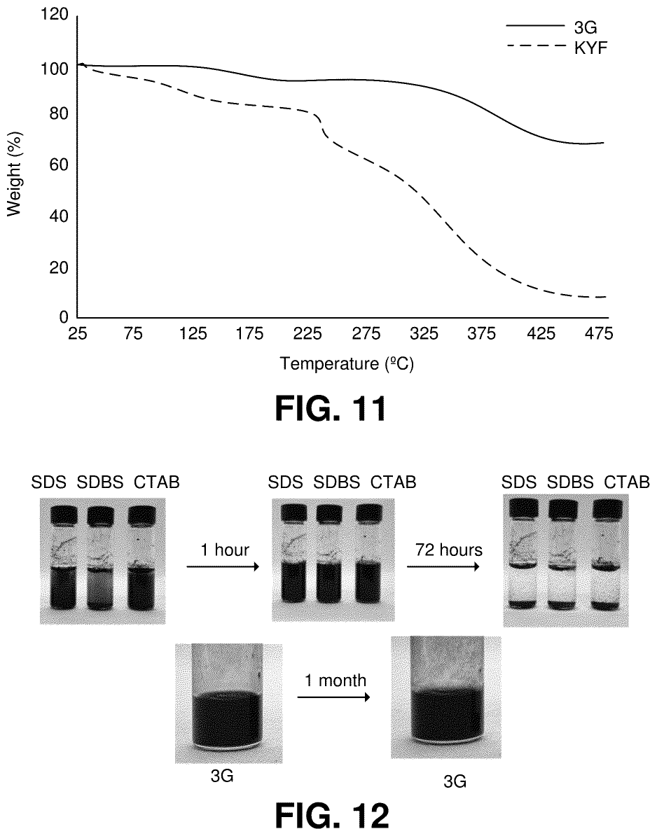

The degree of covalent surface functionalization was further assessed with Raman spectroscopy. Two metrics were used to determine covalent functionalization, a shift in the G-band and a change in I_{D}/I_{G} ratio. Notably, the covalently functionalized graphene, 3G, displayed a Raman shift of approximately 16 cm⁻¹, as well as visual broadening of the G-band compared to bulk graphene powder in 1G (FIG. 3A). The I_{D}/I_{G} ratios of 3G and 1G were 0.76 and 0.47, respectively, indicating a higher level of disorder in 3G attributed to the covalent attachment of KYF molecules (FIG. 3B). In contrast, 2G exhibited an I_{D}/I_{G} value of 0.34, this smaller value is likely due to the disruption of graphene packing upon addition of peptide. In addition, the intensity ratio I_{2D}/I_{G} was quantified to ascertain the extent of graphene layer exfoliation. The recorded I_{2D}/I_{G} values for 1G, 2G and 3G were found to be 0.42, 0.74, and 0.75, respectively, confirming exfoliation of graphene layers after the addition of peptide (FIG. 3C). A thermogravimetric analyzer (TGA) was used to quantify the peptide consumption in the system and estimate the loading of peptide molecules on the graphene surface area. The total weight loss observed upon degradation of peptide on the functionalized graphene was about 30% (FIG. 11), which leads to an estimation of 1.4 peptide molecules per nm². Furthermore, surface coverage was estimated based on multiple potential peptide configurations. The analysis revealed an approximately 56% coverage, forming a relatively dense layer of peptides on the graphene surface. This observation correlates with the heightened stability observed in the aqueous dispersion and is consistent with the observed zeta potential. The disclosed system was compared with conventional surfactants used to disperse graphene in aqueous solutions (SDS, SDBS and CTAB). The disclosed system is stable for weeks without resulting in crashed out graphene aggregates (FIG. 12). In stark contrast, the conventional surfactants formed aggregates in less than 72 hours.

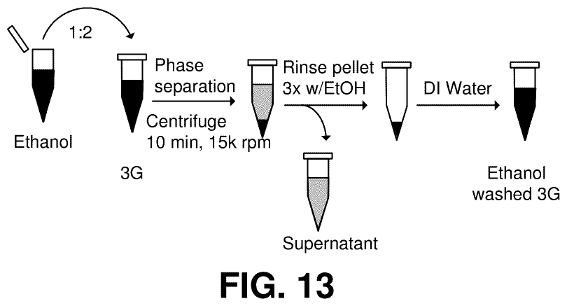

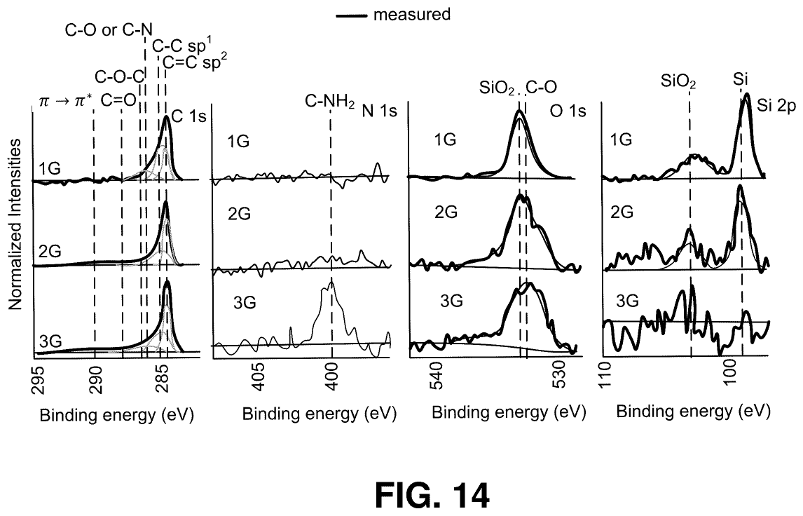

X-ray Photoelectron Spectroscopy (XPS) was used to analyze 1G, 2G and 3G at the molecular level (FIG. 3D). Unwanted binding energies arising from contamination were minimized by subjecting the samples to an extensive ethanol wash procedure to ensure all non-covalent contaminants are removed (FIG. 13). Compared to 1G, a significant increase in intensity was observed in the C Is spectrum for 2G, attributed to the dispersion of graphene, which exposes a larger carbon surface area to the scanning instrument (FIG. 3D). Notably, the effect was higher in the case of 3G due to the additional carbons introduced by the peptide's interaction with graphene (FIG. 3D). Moreover, no peaks were observed for 1G and 2G in the N Is spectra, but only in 3G, validating the presence of peptide nitrogen on the graphene surface with a measurable intensity difference of a C—NH₂ signal (FIG. 3D). Finally, the Ols peak analysis provided valuable insights into the dispersion of the samples. The elevated oxygen intensity observed in 1G can be attributed to the silicon wafer substrate as the binding energy of SiO₂ is ˜533 eV±0.4 eV, while the dispersion facilitated by the peptide in 2G and 3G decreases the peak intensity covering more surface area as seen in optical and SEM images (FIG. 2A and FIG. 2D). In addition, the observed shift of 0.5 eV in 3G supports the proposed engagement of the phenol oxygen in the graphene functionalization, and so the appearance of a C—O bond formation (FIG. 3D). Further evidence to support surface area coverage of oxygen from the substrate in relation to the biofunctionalized graphene was analyzed with the Si 2p spectra of each sample (FIG. 14).

Lastly, to further investigate the versatility and applicability of the disclosed functionalization approach, HYF tripeptide was tested and resulted in a zeta potential value of −31.6 mV mV (FIG. 8), thereby providing a stable dispersion. Several dipeptides were also tested (YR, WY, FY, and LY). The dipeptides containing a hydrophobic amino acid sidechain coupled with tyrosine (WY, FY, LY) resulted in high zeta potential values verifying successful dispersion through hydrophobic interactions with graphene (FIG. 16). This experiment suggests the main dispersing group is the terminal carboxylate, and a second hydrophobic amino acid is needed. An alternative graphene analog with no defects (pristine graphene, p-G) was also studied. Enzymatic oxidation of p-G achieved stability thresholds with zeta potential values of −30.1 mV (FIG. 8).

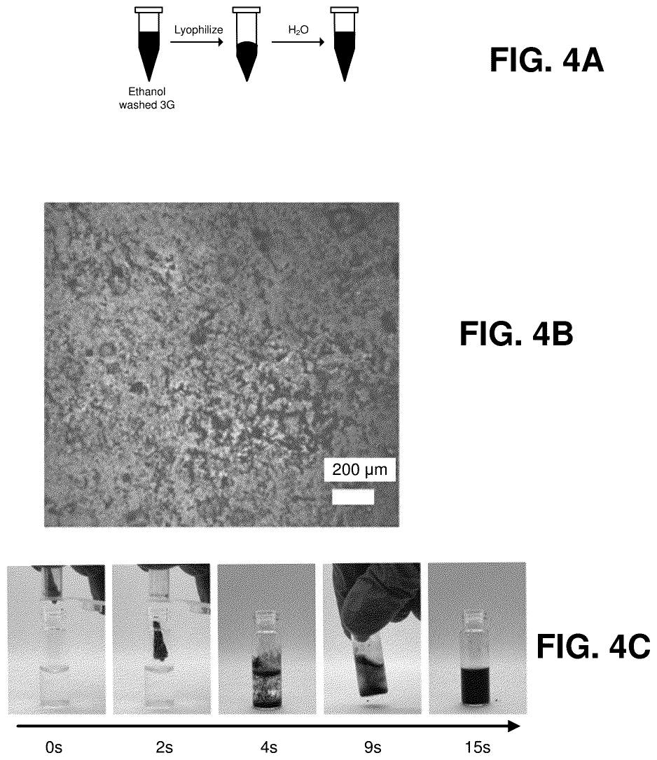

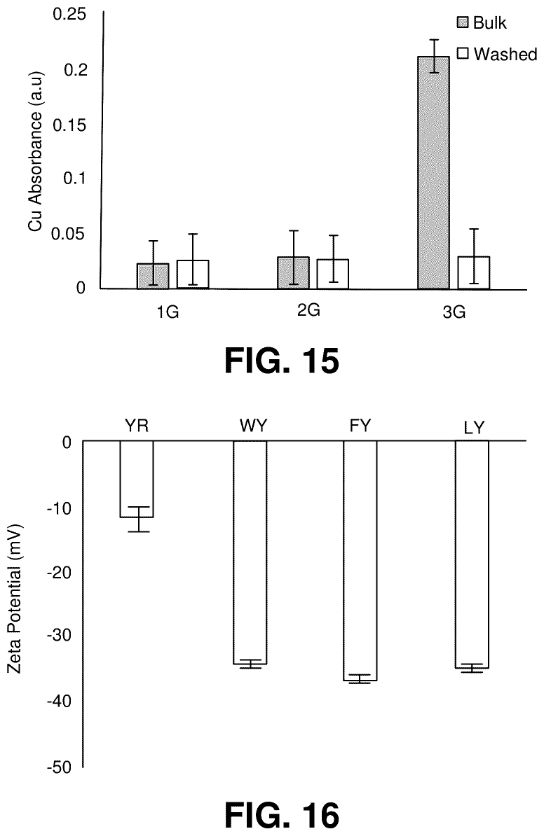

The ability to re-disperse the material after thoroughly washing and freeze-drying the samples was tested, as this serves as a confirmation of the robust covalent bonding interaction established between the peptide and the graphene, giving rise to a storable and readily redispersed formulation. After following the washing procedure, the graphene solution was lyophilized, giving rise to a black powder, and redispersed in DI water (FIG. 4A). Snapshots of the redispersed graphene powder showed the similar behavior of graphene dispersion (FIG. 4B). FIG. 15C shows the materials quick and easy redisperse in water within 15 seconds, without the need for extensive mixing or sonication. This assessment has confirmed the function of tyrosinase in facilitating tyrosine oxidation leading to covalent functionalization of graphene. In order to confirm removal of the enzyme from these graphene preparations post dispersion and washing, a copper assay was employed, a method well-suited for detecting the enzyme's presence as tyrosinase is a metallo-enzyme containing two copper ions in its active site (FIG. 12). As anticipated, bulk 1G and 2G samples exhibited very little absorbance and no significant change of copper content compared to their ethanol washed analogs, while 3G, having tyrosinase in the mixture, displayed a measurable presence of copper in the solution (FIG. 12). Following the ethanol washing step, the sample was effectively cleansed of tyrosinase content, ensuring its near complete removal from the solution, which further confirms that the enzyme protein, beyond its role in catalyzing the covalent functionalization, is not involved in the dispersion process.

While the invention has been described with reference to certain embodiments, it will be understood by those skilled in the art that various changes may be made and equivalents may be substituted for elements thereof to adapt to particular situations without departing from the scope of the disclosure. Therefore, it is intended that the claims not be limited to the particular embodiments disclosed, but that the claims will include all embodiments falling within the scope and spirit of the appended claims.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for forming an aqueous graphene-peptide dispersion, the method comprising:
mixing graphene, a peptide having from two to four residues, water and an oxidant, the peptide having a first residue that is Y, a second residue that is H, Y, W, F, A, V, L, I, P or M, optionally a third residue that is K, H, R, D or E and optionally a fourth residue that is an amino acid, the peptide having an N-terminus that is optionally acetylated and a C-terminus that is optionally amidated, thereby forming a suspension; and
sonicating or vortexing the suspension until an aqueous graphene-peptide dispersion forms.

2. The method as recited in claim 1, wherein the peptide is a tripeptide consisting of the first residue, the second residue and the third residue.

3. The method as recited in claim 2, wherein the tripeptide is KYF.

4. The method as recited in claim 2, wherein the tripeptide is HYF.

5. The method as recited in claim 2, wherein the tripeptide is Ac-KYF.

6. The method as recited in claim 2, wherein the second residue is A, V, L, I, P or M.

7. The method as recited in claim 2, wherein the second residue is H, Y, W or F.

8. The method as recited in claim 2, wherein the third residue is D or E.

9. The method as recited in claim 2, wherein the tripeptide is selected from the group consisting of KYF, HYF, KYY, HYY, DYF, EYF and Ac-KYF.

10. The method as recited in claim 2, wherein the tripeptide is selected from the group consisting of KYF and Ac-KYF.

11. An aqueous graphene-peptide dispersion formed according to the method of claim 1.

12. An aqueous graphene-peptide dispersion formed according to the method of claim 2.

13. The method as recited in claim 1, wherein the peptide is a dipeptide consisting of the first residue and the second residue.

14. The method as recited in claim 13, wherein the second residue is A, V, L, I, P or M.

15. The method as recited in claim 13, wherein the second residue is H, Y, W or F.

16. The method as recited in claim 13, wherein the dipeptide is selected from the group consisting of YR, WY, FY, and LY.

17. An aqueous graphene-peptide dispersion formed according to the method of claim 13.

18. The method as recited in claim 1, wherein the peptide is a tetrapeptide consisting of the first residue, the second residue, the third residue and the fourth residue.

19. The method as recited in claim 1, wherein the oxidant is a tyrosinase enzyme.

20. A method for forming an aqueous graphene-peptide dispersion, the method comprising:
mixing graphene, a tripeptide, water and an oxidizing enzyme, the tripeptide having a first residue that is Y, a second residue that is H, Y, W, F, A, V, L, I, P or M, and a third residue that is K, H, R, D or E, the tripeptide having an N-terminus that is optionally acetylated and a C-terminus that is optionally amidated, thereby forming a suspension; and
sonicating or vortexing the suspension until an aqueous graphene-peptide dispersion forms.
